(19) United States
(12) Patent Application Publication
(10) Pub. No.: US 2015/0284716 A1
(43) Pub. Date: Oct. 8, 2015

(54) METHOD FOR SINGLE CELL SEQUENCING OF MIRNAS AND OTHER CELLULAR RNAS

(71) Applicant: BOARD OF REGENTS, THE UNIVERSITY OF TEXAS SYSTEM, Austin, TX (US)

(72) Inventors: David Corey, Dallas, TX (US); Yongjun Chu, Dallas, TX (US); Bethany Janowski, Dallas, TX (US)

(73) Assignee: BOARD OF REGENTS, THE UNIVERSITY OF TEXAS SYSTEM, Austin, TX (US)

(21) Appl. No.: 14/624,170

(22) Filed: Feb. 17, 2015

Related U.S. Application Data
(60) Provisional application No. 61/941,177, filed on Feb. 18, 2014.

(51) Int. Cl. C12N 15/10; C12Q 1/68

(52) U.S. Cl. CPC C12N 15/1068; C12Q 1/6806

(57) ABSTRACT

Provided herein are methods for single cell sequencing of miRNAs and other low abundance and/or or short cellular RNAs.

### BACKGROUND

1. Field of the Invention

The present disclosure relates generally to the field of molecular biology. More particularly, it concerns methods for sequencing short RNAs from small starting quantities of RNA (e.g., from a single cell).

2. Description of Related Art

RNA sequencing (RNA-Seq) has become a widely-used tool for understanding gene expression (Ozsolak and Milos, 2011). Millions of sequence “reads” can be obtained and subsequent analysis can reveal fine details of gene expression and regulation. Depending on the size of the starting RNA used, RNA-Seq can generally be divided into two categories: long RNA-Seq and small RNA-Seq. For sequencing long RNA fragments (>200 bases), reverse transcription using random primers to make cDNA is often favored and amounts as low as 10-100 pg of RNA can be analyzed (Ramsköld et al., 2012). This method allows partial investigation of the transcriptome of single cells (Tang et al., 2009; Tang et al., 2011; Xue et al., 2013; Shalek et al., 2013) but is not amenable to the sequencing of small RNAs (<40 nt) (Adiconis et al., 2013). The study of miRNAs, endogenous trans-acting siRNAs, repeat-associated siRNAs, piRNAs, and heavily-fragmented long RNAs derived from various techniques requires much larger amounts of material, and the need for more material can be an obstacle for research (Adiconis et al., 2013).

For small RNA-Seq library preparation, it is necessary to sequentially ligate adaptors to the RNA 3′- and 5′-ends. This strategy is used by all protocols including the widely-used Illumina TruSeq small RNA sequencing protocol (Borges-Rivera et al., 2010). While effective in many cases, the method requires two successful ligations and may be sensitive to structure at the termini where adaptor ligation must occur. RNAs with less than three unstructured bases at the 3′-end are not efficiently ligated (Zhuang et al., 2012). RNA molecules that have secondary structure near their termini or that are prone to be associated with other RNA molecules are also not well detected by these methods (Zhuang et al., 2012). Because of these challenges, intermolecular RNA-RNA ligations leave many input RNA sequences unreacted. As such, manufacturers of standard small RNA-Seq protocols suggest using greater than 100 ng of small cellular RNA starting material for optimal results.

The need for greater than 100 ng small RNA starting material is a problem for many applications where starting material is limited (Adiconis et al., 2013; McCormick et al., 2010). These applications include analysis of extracellular RNA (Esther et al., 2012), examination of relatively small numbers of cells, clinical samples, RNA isolated from cellular compartments, such as mitochondria (Mercer et al., 2011) or nuclei, and RNA isolated after immunoprecipitation protocols, such as CLIP-Seq (Chi et al., 2009; Hafner et al., 2010). In at least these instances, the inefficiency of the ligation step will limit the total number of reads. Furthermore, secondary structure at some termini will block ligation and limit the coverage of sequences causing them to be overlooked.

There is a need for straightforward methodology that can be readily adopted by researchers accustomed to standard RNA-Seq protocols and platforms, that has the necessary sensitivity for small (<200 nucleotide) nucleotide fragments, and that demonstrates at least a similar quality of sequencing output relative to standard methods.

### SUMMARY

Provided herein are methods for obtaining sequences of RNA from small quantities of RNA (e.g., RNA from a single cell), especially short RNAs (e.g., miRNAs). These methods avoid the challenges inherent in intermolecular ligation while working at temperatures that reduce secondary structure and allow more uniform recognition of fragment termini.

In one embodiment, a method is provided for preparing an RNA sample for sequencing comprising: (a) obtaining a sample comprising RNA molecules; (b) self-ligating each RNA molecule in the sample to form circular RNA; (c) hybridizing a first set of random primers to the circular RNA; (d) extending the first set of random primers hybridized to the circular RNA to form cDNA; (e) self-ligating the cDNA to form a circular cDNA; (f) hybridizing a second set of random primers to the circular cDNA; and (g) extending the second set of random primers hybridized to the circular cDNA to form double-stranded cDNA. In some aspects, steps (c) and (d) and/or steps (f) and (g) may be performed simultaneously. In other aspects, steps (c) and (d) and/or steps (f) and (g) may be performed sequentially in the absence of exogenous manipulation.

In some aspects, the self-ligating of step (b) may comprise treating the at least one RNA with a template-independent, single-stranded RNA ligase, such as, for example, CircLigase II, RtcB, or T4 RNA ligase. In certain aspects, the self-ligating of step (e) may comprise treating the cDNA with a template-independent, single-stranded DNA ligase, such as, for example, CircLigase or CircLigase II.

In certain aspects, the first set of random primers of step (c) and/or the second set of random primers of step (f) may be random hexamers. In one aspect, the second set of random primers of step (f) may be nuclease-resistant RNA primers.

In one aspect, the extending of step (d) may comprise performing reverse transcription. In one aspect, the extending of step (g) may comprise performing a polymerization reaction with Phi29 polymerase, Bst DNA polymerase, large fragment, or Bst 2.0 DNA polymerase (New England Biolabs). In a further aspect, the polymerization reaction of step (g) may comprise trehalose.

In one aspect, the method may comprise (h) fragmenting the double-stranded cDNA. In some aspects, fragmenting may comprise sonication, enzymatic digestion, or metal-assisted hydrolysis.

In some aspects, the RNA molecules of step (a) may be single-stranded. In certain aspects, the RNA sample of step (a) may comprise or consist essentially or less than 100 ng, 50 ng, 1 ng, 500 pg, 250 pg, 100 pg, 50 pg, but having a minimum amount of at least 10 pg, 10-500 pg, 10-250 pg, 10-200 pg, or 10-100 pg of RNA. In one aspect, the RNA sample may comprise RNA obtained from a single cell. In other aspects, the RNA sample of step (a) may comprise or consist essentially of RNA molecules less than 200 nt, 100 nt, 50 nt, or 20 nt, 20-750 nt, 100-600 nt, 200-500 nt, or 100-200 nt in length. In yet other aspects, the RNA sample of step (a) may consist of RNA molecules less than 200 nt, 100 nt, 50 nt, or 20 nt in length, but having a minimum length of 20 nt.

In a further aspect, the method may comprise (i) ligating adaptors into the 5′ and 3′ ends of the fragmented cDNA to form adapted cDNA. In one aspect, the fragmented cDNA may be subjected to end repair A-base addition prior to ligation. In one aspect, the adaptors may comprise y-shaped adaptors.

In yet a further aspect, the method may comprise (j) amplifying the adapted cDNA of step (i) thereby producing a sequencing library. In some aspects, amplifying may comprise performing PCR. The PCR may be performed using indexed or barcoded primers. In this aspect, the primers may comprise a known sequence.

In yet a further aspect, the method may comprise (k) obtaining sequencing data for the sequencing library. The sequencing data may be obtained using any known sequencing platform, such as, for example, the Illumina HiSeq2000 platform. In one aspect, the method may comprise (1) identifying the original RNA sequence by aligning to a reference. The aligning may comprise performing an expanding-then-aligning algorithm. The expanding-then-aligning algorithm may comprise the computer program listings of Appendix A-E.

In one embodiment, a method is provided for preparing an RNA sample for sequencing comprising: (a) obtaining a sample comprising RNA molecules; (b) self-ligating each RNA molecule in the sample to form circular RNA; (c) hybridizing a first set of random primers to the circular RNA, wherein the first set of random primers comprises a 5′ adaptor of known sequence; (d) extending the first set of random primers hybridized to the circular RNA to form cDNA; (e) hybridizing a second set of random primers to the cDNA, wherein the second set of random primers comprises a 3′ adaptor of known sequence; and (f) extending the second set of random primers hybridized to the cDNA. In some aspects, steps (c) and (d) and/or steps (e) and (f) may be performed simultaneously. In other aspects, steps (c) and (d) and/or steps (e) and (f) may be performed sequentially in the absence of exogenous manipulation. In one aspect, the extending of step (d) may comprise performing reverse transcription.

In some aspects, the self-ligating of step (b) may comprise treating the at least one RNA with a template-independent, single-stranded RNA ligase, such as, for example, CircLigase II, RtcB, or T4 RNA ligase.

In certain aspects, the random portions of the first set of random primers comprising a 5′ adaptor of known sequence of step (c) and second set of random primers comprising a 3′ adaptor of known sequence of step (e) may be random hexamers. In other aspects, the adaptor portions of the first set of random primers comprising a 5′ adaptor of known sequence of step (c) and second set of random primers comprising a 3′ adaptor of known sequence of step (e) may be different. In one aspect, the first set of random primers of step (c) and/or the second set of random primers of step (e) may be nuclease-resistant RNA primers.

In some aspects, the RNA molecules of step (a) may be single-stranded. In certain aspects, the RNA sample of step (a) may comprise less than 100 ng, 50 ng, 1 ng, 500 pg, 250 pg, 100 pg, 50 pg, or 10 pg of RNA. In one aspect, the RNA sample may comprise RNA obtained from a single cell. In other aspects, the RNA sample of step (a) may comprise RNA molecules less than 200 nt, 100 nt, 50 nt, or 20 nt in length. In yet other aspects, the RNA sample of step (a) may consist essentially of RNA molecules less than 200 nt, 100 nt, 50 nt, or 20 nt in length.

In yet a further aspect, the method may comprise (g) amplifying the cDNA of step (f) thereby producing a sequencing library. In some aspects, amplifying may comprise performing PCR. The PCR may be performed using indexed or barcoded primers. In this aspect, the primers may comprise a known sequence.

In yet a further aspect, the method may comprise (h) obtaining sequencing data for the sequencing library. The sequencing data may be obtained using any known sequencing platform, such as, for example, the Illumina HiSeq2000 platform. In one aspect, the method may comprise (i) identifying the original RNA sequence by aligning to a reference. The aligning may comprise performing an expanding-then-aligning algorithm. The expanding-then-aligning algorithm may comprise the computer program listings of Appendix A-E.

In one embodiment, a kit is provided comprising a single-stranded RNA ligase, a reverse transcriptase, and a DNA polymerase. In other aspects, the kit may also comprise a single-stranded DNA ligase, a DNA ligase, Y-shaped DNA adaptors, trehalose. In yet other aspects, the kit may comprise random hexamer primers, DNA primers that hybridize to an adaptor sequence, deoxyribonucleotides, and at least one buffer. In yet other aspects, the kit may comprise software that identifies the original RNA sequence by aligning to a reference. The software may perform an expanding-then-aligning algorithm. The expanding-then-aligning algorithm may comprise the computer program listings of Appendix A-E. In yet other aspects, the kit may comprise software that identifies protein binding sites within the original RNA sequence.

In certain aspects, the single-stranded RNA ligase may be CircLigase II, RtcB, or T4 RNA ligase. In certain aspects, the single-stranded DNA ligase is CircLigase or CircLigase II. In some aspects, the DNA polymerase may be Phi29 DNA polymerase, Bst DNA polymerase, large fragment, or Bst 2.0 DNA polymerase (New England Biolabs).

In some aspects, the random hexamer primers may be nuclease-resistant RNA primers. In one aspect, a portion of the random hexamer primers may comprise a 5′ adaptor of known sequence. In another aspect, a portion of the random hexamer primers may comprise a 3′ adaptor of known sequence. In these aspects, the kit may comprise multiple, individually-contained primer samples, such as, for example, random hexamers comprising a 5′ adaptor of known sequence and random hexamers comprising a 3′ adaptor of known sequence.

As used herein, the term “consisting essentially of” with regard to a nucleic acid sample means that the sample does not contain any material that does not fit the identified criteria, at least not at a readily detectable level. For example, a sample that consists essentially of RNA molecules less than 100 nt in length can mean that based on standard detection methods (e.g., gel electrophoresis or bioanalyzer analysis) the sample only contains negligible quantities of RNA molecules greater than 100 nt in length, preferably at such levels as cannot be detected by the standard detection methods. However, one of skill in the art will recognize that such a sample may contain longer RNA molecules, DNA molecules, proteins, or other cellular components, but only in such quantities as to not materially affect the basic characteristics of the sample. The term “consisting essentially of” is not meant to exclude the inclusion of buffers, salts, and other inert chemicals from being present in the sample.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-F: RNA-circularization based RNA sequencing (RC-Seq). (FIG. 1A) Scheme showing how a sequencing library is made in RC-Seq. (FIG. 1B) Efficient intramolecular circularization of synthetic RNAs (randomized 20mer oligonucleotides; L-20) by CircLigase II ssDNA ligase and removal of remaining linear RNA by RNase R. Lane 1: linear single-stranded L-20 RNA; lane 2: linear L-20 RNA treated with 5 U RNase R; lane 3: linear L-20 RNA treated with 20 U RNase R; lane 4: circularized product of L-20 RNA (C-20); lane 5: circularized product of L-20 RNA (C-20) treated with 5 U RNase R; lane 6: circularized product of L-20 RNA (C-20) treated with 20 U RNase R. (FIG. 1C) cDNA products generated after reverse transcription of circular product of 20 nt (C-20), 40 nt (C-40), and 60 nt (C-60) randomized L-20, L-40, and L-60 RNAs, respectively. (FIG. 1D) Scheme showing the expanding-then-alignment approach (see the computer program listings Appendix A-E) used in data processing. (FIG. 1E) Expanding-then-alignment approach reliably finds the genomic location of an original RNA molecule. Percentage of correctly aligned reads from regular alignment approach and expanding-then-alignment approach are comparable. Five different groups of reads, 20 nt, 40 nt, 60 nt, 80 nt and 100 nt, were used in the simulation. For each group, 5000 reads were randomly selected from human genome (hg19). (FIG. 1F) Regular alignment approach and expanding-then-alignment approach showing comparable error rates. The percentages of incorrectly aligning reads are close to each other for both methods. The input data was the same as that in FIG. 1E.

FIGS. 2A-B: RC-Seq method performed better than TruSeq while requiring much less starting material and generating deeper sequencing depth. (FIG. 2A) RC-Seq yielded more unique reads than commercial the TruSeq kit when 100 ng of starting RNA was used for both. (FIG. 2B) RC-Seq yielded a large number of unique reads even when only 1 ng of RNA was used as the starting material.

FIGS. 3A-D: The application of RC-Seq method in sequencing human AGO2-associated clipped RNAs. The clipped RNA was isolated following a PAR-CLIP protocol. (FIG. 3A) P³² image showing no noticeable ligation occurring between clipped RNA and a preadenylated 3′-adaptor. (FIG. 3B) P³² image showing efficient intramolecular circularization of clipped RNA. Lane 1: clipped RNA; lane 2: clipped RNA treated with RNaseR; lane 3: circularized clipped RNA treated with RNaseR. (FIG. 3C) Mutation rates in the aligned data. (FIG. 3D) Genomic annotation of identified significant AGO2-bound clusters. The Mi-CLIP program (Wang et al., 2014) was used to predict the AGO2 binding sites.

FIGS. 4A-B: Modified RC2-seq for picograms of RNA or single cell RNA sequencing. (FIG. 4A) Scheme showing the workflow of RC2-seq. (FIG. 4B) Agarose gel (1%) image demonstrating ultra-high sensitivity and specificity of RC2-Seq library preparation. As low as 10 pg of RNA (single-cell amount of RNA; tested RNA was a random 40 nt mixture, RD-40-N9) successfully amplified to yield greater than 1 μg double-stranded DNA, enough for sequencing library preparation. No amplification product appeared in the no-template lane.

FIG. 5: Scheme showing improved RC3-Seq library preparation.

FIG. 6: High quality libraries generated with low input small RNA. The input RNA was 40 nt randomized synthetic RNA, RD-40-N9 (Table 1). Lane 2-5: 10 ng, 1 ng, 100 pg, 10 pg of RNA input. Lane 6: no RNA input control.

### DESCRIPTION OF ILLUSTRATIVE EMBODIMENTS

Novel, strand-specific small RNA library construction methods are provided herein. The present methods are useful for sequencing short RNAs, especially from a single cell. In these methods, only picograms of RNA are needed, and nearly all isolated RNA species can be efficiently converted into a sequencing library. This method includes a highly-efficient intramolecular RNA circularization step and a random priming step to generate full-length cDNA. Data can be obtained with much smaller quantities of RNA while maintaining the same or better quality as data commonly obtained using standard RNA-adaptor intermolecular ligation-based methods (e.g., Illumina TruSeq protocol). Tradition RNA-Seq protocols require adaptor ligation (both 3′ and 5′) during library preparation. However, with short RNA molecules, the efficiency of even highly optimized ligation reactions can be extremely low, and RNA-RNA ligation steps also produce multiple byproducts. Furthermore, these methods require at least 100 ng of starting material, which for small RNA is difficult to acquire.

Due to the present methods' high sensitivity and simplicity, these methods are ideal for situations when obtaining sufficient RNA is a challenge and sequencing depth is not adequate. These methods will find wide applications in small RNA-Seq, particularly when sufficient RNA cannot be obtained to construct a sequencing library. RNA isolated from HITS-CLIP (also known as CLIP-Seq), PAR-CLIP, or a single cell, or highly-structured RNAs are ideal candidates. However, these approaches may also be used for longer RNA (>200 nt) and DNA sequencing.

CLIP-Seq is a genome-wide means of mapping protein-RNA binding sites. CLIP-Seq is similar to ChIP-Seq, except that proteins bound to RNA are immunoprecipitated and the RNA fragments then sequenced. To construct CLIP-Seq libraries, cell lysates and/or nuclear lysates are prepared and treated with DNAse. The sample is then incubated with an antibody to the desired RNA-binding protein of interest, followed by UV crosslinking. Then, RNA-protein complexes are immunoprecipitated, followed by RNAse treatment, electrophoresis of IP material in an SDS-PAGE gel, excision of a specific RNA-protein band, and RNA extraction. Exemplary methods for performing CLIP-Seq are described in Yeo et al. (2009); Zhang and Darnell (2011); Jensen and Darnell (2008); Licatalosi et al. (2008); Ule et al. (2005); and Ule et al. (2003).

PAR-CLIP is similar to CLIP-Seq except that it employs the photoreactive thionucleosides, 4-thiouridine and 6-thioguanosine, to increase the crosslinking efficiency between protein and RNA and to provide near-nucleotide resolution of the RNA-binding site (Hafner et al., 2010).

### I. DEFINITIONS

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

A “nucleic acid molecule of interest” can be a single nucleic acid molecule or a plurality of nucleic acid molecules. Also, a nucleic acid molecule of interest can be of biological or synthetic origin. Examples of nucleic acid molecules include double-stranded molecules, single-stranded molecules, genomic DNA, cDNA, RNA, amplified DNA, a pre-existing nucleic acid library, etc. The term “double-stranded molecule” as used herein refers to a molecule that is double stranded at least in part. A nucleic acid molecule of interest may be subjected to various treatments, such as repair treatments and fragmenting treatments. Fragmenting treatments include mechanical, sonic, chemical, enzymatic, degradation over time, etc. Repair treatments include nick repair via extension and/or ligation, polishing to create blunt ends, removal of damaged bases such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 5-100 “cycles” of denaturation and replication.

“Incorporating,” as used herein, means becoming part of a nucleic acid polymer.

“Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.”

“Primer” as used herein refers to a single-stranded oligonucleotide or a single-stranded polynucleotide that is extended by covalent addition of nucleotide monomers during amplification. Often, nucleic acid amplification is based on nucleic acid synthesis by a nucleic acid polymerase. Many such polymerases require the presence of a primer that can be extended to initiate nucleic acid synthesis.

The term “sequencing primer” as used herein, refers to a specific nucleotide sequence configured to initiate amplification for high throughput sequencer platforms, including but not limited to Illumina, SOLiD or 454.

The term “barcode” as used herein, refers to any unique, non-naturally occurring, nucleic acid sequence that may be used to identify the originating genome of a nucleic acid fragment. The barcode sequence provides a high-quality individual read of a barcode associated with a sample such that multiple different samples can be sequenced together.

The term “next-generation sequencing platform” as used herein, refers to any nucleic acid sequencing device that utilizes massively parallel technology. For example, such a platform may include, but is not limited to, Illumina sequencing platforms. Other examples include Roche 454, Pacific Bioscience, Ion Torrents, Harvard Polonator, ABI Solid or other similar instruments in the field. Classic sequencing approaches, such as Sanger sequencing can be used; however, the true power in the technology is to be able to sequence a larger number of sequences from single cells simultaneously.

“Low abundance” as used herein refers to an RNA species that comprises less than 1% of the RNA species in a population of RNAs. Such a low abundance RNA species may comprise less than 1%, 0.75%, 0.5%, 0.25%, 0.1%, 0.05%, or 0.01%, or any number derivable therein, of the RNA species present in a population of RNAs.

“Short” or “small” RNA as used herein refers to an RNA less than 200 nucleotides in length. Such an RNA may consist of less than 200 nt, 150 nt, 100 nt, 90 nt, 80 nt, 70 nt, 60 nt, 50 nt, 40 nt, 30 nt, 20 nt, or 10 nt, or any number derivable therein. In a sample comprising a population of short RNAs, the sample may contain RNAs of various lengths, such as between 10 nt and 200 nt, 10 nt and 100 nt, 20 nt and 150 nt, 20 nt and 100 nt, 20 nt and 50 nt, or any range derivable therein. Non-limiting examples of short RNAs include miRNA, piRNA, rasiRNA, siRNA, endogenous transacting siRNA, repeat-associated siRNA, and heavily-fragmented long RNAs.

A “small quantity” of RNA as used herein refers to a quantity of RNA less than 100 ng, 50 ng, 10 ng, 1 ng, 500 pg, 250 pg, 100 pg, 50 pg, or 10 pg, or any number derivable therein. A small quantity of RNA may be containing in a range of volumes of a suitable liquid (e.g., dH₂O, a buffer, ethanol, etc.), such as, for example 1-10 μl, 1-100 μl, 1-1000 μl, 10-200 μl, 10-100 μl, or 100-1000 μl, or any range derivable therein. A small quantity of RNA may be in lyophilized form. Non-limiting examples of sources of small quantities of RNA include RNA isolated from immunoprecipitation, such as CLIP RNA, RNA extracted from a single cell, extracellular RNA, or RNA isolated from intracellular organelles, such as mitochondria and nuclei.

The term “in the absence of exogenous manipulation” as used herein refers to there being modification of a DNA molecule without changing the solution in which the DNA molecule is being modified. In specific embodiments, it occurs in the absence of the hand of man or in the absence of a machine that changes solution conditions, which may also be referred to as buffer conditions. In further specific embodiments, changes in temperature occur during the modification.

The term “ligase” as used herein refers to an enzyme that is capable of joining a hydroxyl terminus of one nucleic acid molecule to a phosphate terminus of either the same or a second nucleic acid molecule to form either a circular nucleic acid or a single linear molecule. Such enzymes may use RNA and/or DNA as a substrate. Such enzymes may join a 3′ hydroxyl terminus and a 5′ phosphate terminus. Alternatively such enzymes may join a 5′ hydroxyl terminus and a 3′ phosphate terminus. Enzymatic digestion or metal-assisted hydrolysis of RNAs yields two types of RNA fragments: those with a 5′-OH/3′-PO⁴⁻ structure and those with a 5′-PO⁴⁻/3′-OH structure. Linear RNAs was a 5′-PO⁴⁻/3′-OH structure can be circularized by, for example, CircLigase II ssDNA ligase. Linear RNAs with a 5′-OH/3′-PO⁴⁻ structure can be circularized by specific ligases available for this purpose (Chakravarty et al., 2012). Since both types of RNAs can be circularized, almost all cellular RNAs can be sequenced by the methods disclosed herein.

The term “kit” as used herein refers to one or more suitably aliquoted compositions or reagents for use in the methods of the present disclosure. The components of the kits may be packaged either in aqueous or lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the reagent containers in close confinement for commercial sale. Such containers may include injection or blow molded plastic containers into which the desired vials are retained, for example.

### II. PREPARATION OF SEQUENCING LIBRARIES

Adapters for use in the disclosure will generally include a double-stranded region adjacent to the “ligatable” end of the adapter, i.e. the end that is joined to a target polynucleotide in the ligation reaction. The ligatable end of the adapter may be blunt or, in other embodiments, short 5′ or 3′ overhangs of one or more nucleotides may be present to facilitate/promote ligation. The 5′ terminal nucleotide at the ligatable end of the adapter should be phosphorylated to enable phosphodiester linkage to a 3′ hydroxyl group on the target polynucleotide.

An adapter may contain a modified component such as, for example, a modified nucleotide or a modified bond. In one embodiment, the modified nucleotide or bond differs in at least one respect from deoxycytosine (dC), deoxyadenine (dA), deoxyguanine (dG) or deoxythymine (dT). Where the adapter is DNA, examples of modified nucleotides include ribonucleotides or derivatives thereof (for example: uracil (U), adenine (A), guanine (G) and cytosine (C)), and deoxyribonucleotides or derivatives thereof such as deoxyuracil (dU) and 8-oxo-guanine. Where the adapter is RNA, the modified nucleotide may be a dU, a modified ribonucleotide or deoxyribonucleotide. Examples of modified ribonucleotides and deoxyribonucleotides include abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin Bl-fapy-guanine, fapy-adenine, hypoxanthine, methyl-fapy-guanine, methyltartonylurea and thymine glycol. Examples of modified bonds include any bond linking two nucleotides or modified nucleotides that is not a phosphodiester bond. An example of a modified bond is a phosphorothiolate linkage.

The adapter may have a blunt-ended terminus or an overhang at either the 5′ or 3′ end. Where the terminal region has an overhang, it may be an overhang of a single base such as generated by the terminal transferase activity of Taq DNA polymerase, or more than one base, for example, sequences complementary to the cohesive ends generated by many restriction endonucleases, including, for example EcoRI, EcoRII, BamHI, Hind111, Taq1, Not1. Ligation of adapters to target polynucleotides such as fragments of DNA in a library which have a single base overhang may be enhanced by the use of a small molecule enhancer.

Ligation may alternatively be enhanced by polishing staggered ends of a duplex polynucleotide using a mixture of polymerases where one of the polymerases is a thermostable polymerase with 3′-5′ exonuclease activity. The mixture can include, for example, T4 DNA polymerase and an archeael polymerase. A mixture of polymerases for polishing DNA ends can be used to prepare any type or number of duplex polynucleotides for ligation for example to y-shaped adapters.

The 5′ end of an adapter may be modified to aid ligation of the adapter to a polynucleotide of interest. Modifications to the 5′ end of the adapter ligation include phosphorylation and adenylation. Modifications may be achieved by any means known in the art including methods comprising the use of T4 polynucleotide kinase for phosphorylation and T4 DNA ligase for adenylation. Modifications such as the incorporation of phosphothioate linkages may also be added to the 5′ and/or 3′ end of the adapter to resist exonuclease degradation.

In some embodiments, prior to ligation of the adaptor, the nucleic acids in a sample can be phosphorylated and/or adenylated. Adenylation can provide an adenosine overhang on the 3′ end of a nucleic acid. A second nucleic acid with a thionine 3′ overhang can then be ligated to the first nucleic acid by TA ligation.

The ligation of adapters to polynucleotide targets may be used in the preparation of polynucleotide libraries. A polynucleotide library may contain non-identical polynucleotides wherein at least one member of the library must contain at least one polynucleotide consisting of a sequence which differs by at least one nucleotide from one or more polynucleotides in the library.

Y-shaped adapters and double-stranded DNA universal adapters with internal mismatches have been developed to add known primer sites to DNA of unknown sequence. These Y-adapters share the property of having two separate strands of DNA to form double-stranded and single-stranded regions (see U.S. Pat. No. 7,741,463, which is incorporated herein by reference in its entirety). The separate strands of the double-stranded adapters are ligated to each end of a target sequence and a primer pair is added to the ligated DNA. One primer anneals to a sequence in an adapter at one end of the target DNA and the other primer in the pair anneals to a sequence on the complementary strand of the adapter at the other end of the target DNA.

A primer may include a 5′ modification, such as an inverted base (e.g. 5′-5′ linkage); one or more phosphothioate bonds to prevent 5′-3′ exonuclease-degradation or unwanted ligation products; a fluorescent entity such as fluorescein to aid in quantification of amplification product; or a moiety, such as biotin to aid in separation of amplification product from solution.

The adapter may contain one or more primer-associated sequences within the adapter. The forward primer site hybridizes to one or more short oligonucleotides, or forward primers. The reverse primer site has a reverse complement that hybridizes to a reverse primer. The forward and reverse primer sequences may be at least about 10 nucleotides in length and located within the single-stranded y-region and/or the double-stranded region of the adapter.

Adapters may additionally include sequence identifiers such as barcodes. Barcodes are preferably a sequence which is rarely found in nature. Barcode sequences may be used to identify and isolate selected polynucleotides as well as to streamline downstream data analysis. A barcode can be assigned to identify specific samples, experiments or lots. Barcode sequences may be at least 2 nucleotides in length and generally no more than about 15 nucleotides in length. This provides resolution for 2⁴-15⁴ different libraries in a single mixture. Barcodes can be used, for example, to isolate adapter-ligated polynucleotides using, for example, oligonucleotide probes.

Barcodes can be used in downstream data analysis. For example, where multiple samples comprising DNA sequences from different species are processed simultaneously, samples containing species-specific unique identifying sequences can be extracted from the raw data based on the presence of the identifier and compared to the reference genome corresponding to the species indicated in the identifying sequence. The unique identifying sequences can also be used within a quality assurance protocol, including use as a means for tracking samples through multiple reactions, personnel or processing locations.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Cell Culture and Synthetic Oligonucleotides.

T47D cells (American Type Culture Collection) were maintained in RPMI-1640 media supplemented with 10% (v/v) FBS, 0.5% (w/v) nonessential amino acids, 0.4 units/mL bovine insulin (all reagents from Sigma). Cells were cultured at 37° C. and 5% (v/v) CO₂. All synthetic RNAs, primers for generating cDNA, and PCR primers were obtained from Integrated DNA Technologies and PAGE purified. The sequences are listed in Table 1.

Endogenous Small RNA Sample Preparation.

Fifteen large dishes (150 cm²) of T47D cells were dissolved in 20 ml of TriZol (Sigma) and total RNA was isolated according to standard TriZol RNA isolation procedure (Sigma). RNA was loaded on a 15% denaturing polyacramide gel and RNA bands located between 40 nt and 15 nt molecular markers were excised and eluted with 0.3 M Na acetate (pH 5.5) containing RNase-In (Promega, final 50 U/ml) overnight at 4° C. The small RNA pellet was isolated by phenol extraction and ethanol precipitation. The RNA pellet was dissolved in water and quantitated by Nanodrop (Fisher Scientific).

PAR-CLIP Sample Preparation.

T47D cells were incubated in fresh media containing 4-thiouridine (Sigma) at 100 μM. Media was removed 14 h later and cells were washed once with Dulbecco's phosphate buffered saline (Sigma) and UV-irradiated at 365 nm with an energy of 300 mJ/cm² on ice. Nuclei were isolated by first incubating the cells in hypotonic lysis buffer (10 mM Tris.HCl pH7.4, 10 mM NaCl, 3 mM MgCl₂, 0.5% NP-40, lx complete protease inhibitor (Roche), 0.5 mM DTT, and 50 U/ml Promega RNase-In) twice for 5 min each on ice (Chu et al., 2010). The supernatant was removed after centrifugation at 500×g for 5 min at 4° C. The crude nuclei were washed once with this hypotonic buffer to get pure nuclei. The nuclei were then suspended in nuclear lysis buffer (150 mM KCl, 20 mM Tris.HCl 7.4, 1.5 mM MgCl₂, 0.5% NP-40, lx complete protease inhibitor, 0.5 mM DTT, and 50 U/ml Promega RNase-In) for 10 min on ice. After vigorous vortexing and pipetting, nuclei were freeze-thawed three times in liquid nitrogen and a 22° C. water bath. The mixture was then subjected to sonication on ice using an Ultrasonic Homogenizer (20% power for 30 s, Model 150V/T, Biologics, Inc.). Insoluble material was removed by centrifugation at maximum speed for 15 min at 4° C. Nuclear extracts were quickly frozen in liquid nitrogen and stored at −80° C.

The AGO2 immunoprecipitation and clipped RNA isolation were carried out based on the original PAR-CLIP protocol (Hafner et al., 2010) except that RNase I was used instead of RNase T1 to avoid potential sequence biases generated and an anti-AGO2 antibody (Sigma) recognizing endogenous AGO2 was used (Chu et al., 2010).

RNA Circularization.

RNA, including synthetic RNA, naturally occurring miRNA, and clipped RNA, were circularized with CircLigase™ II ssDNA Ligase (Epicentre) at 60° C. for 1 h in a 20 μl reaction volume containing 2 μl 10× reaction buffer, 1 μl 50 mM MnCl₂ (Epicentre), 4 μl 5 M Betaine (Epicentre) and 1 μl Ligase. To remove the remaining linear RNA, 2.3 μl of 10× RNase R buffer (Epicentre) and 1 μl of RNase R (20 U, Epicentre) was added to the reaction mixture. The RNase R digestion was carried out at 37° C. for 10 min. After the digestion, an oligo purification column (Zymo Research, Oligo Clean & Concentrator) was used to isolate the circularized RNA by following the producer's instructions. Purified RNA was eluted with nuclease-free water.

RC2-Seq Library Preparation.

Generating the complementary DNA (cDNA) strand from the circularized RNA was performed first. Thus, to a circularized RNA solution was added 2 μl 100 μM cDNA primer (Phos-NNNNNN), 1 μl 10 mM dNTP solution (containing 10 mM dATP, 10 mM dGTP, 10 mM dCTP and 10 mM dTTP) and H₂O to make a total of 12 μl. Following heating of the solution at 65° C. for 5 min, the solution was cooled directly on ice for at least 1 min. To this solution was added 4 μl 5× superscript II reaction buffer, 2 μl 0.1 M DTT, 1 μl RNase-Out and 1 μl Superscript II (Life Technologies). The solution was mixed gently and placed on a thermal cycler at 25° C. for 15 min, then 42° C. for 2 h and finally 70° C. for 15 min to deactivate the enzyme. The cDNA was then column-purified (Zymo Research, Oligo Clean & Concentrator) and eluted with 15 μl H₂O. To this cDNA solution was added 2 μl CircLigase buffer (10×), 1 μl 1 mM ATP, 1 μl MnCl₂ and 1 μl CircLigase ssDNA Ligase (Epicenter, 100 U/μl). The cDNA circularization was carried out at 60° C. for 2 h. To this cDNA reaction mixture (20 μl), 6 μl Phi29 DNA polymerase buffer (10×), 3 μl dNTP (25 mM each), 2 μl 6R2S (rNrNrNrN*rN*rN, 200 μM), 1 μl DTT (0.1 M), 20 μl Trehalose (Sigma, made to 1.2 M with H₂O), 1 μl inorganic pyrophosphatase (20 U), 3 μl Phi29 polymerase (Epicentre, 100 U/μl) and H₂O to make 60 μl in total volume was added. The rolling circle amplification was carried out for 12 h at 30° C. and then 70° C. for 15 min to deactivate the enzyme. Zymo Genomic DNA column was used to isolate long double-stranded DNA product (>10 kb). The eluted pure dsDNA was fragmented by Covaris sonicator to the size range of from 200 to 500 bp. The DNA fragments were then repaired at both 5′ and 3′ ends, subjected to adenosine addition and Y-shape adaptor ligation, by following the instructions of the Kapa DNA sequencing library preparation kit (Kapa Biosystems). The indexes were incorporated into the product by PCR, which was generally performed with 5-10 cycles. All the sequences used are listed in Table 1. The crude PCR product was purified by Agencout AMPure XP magnetic beads (Beckman Coulter) using a 1:1 volume ratio. The final PCR product was eluted with H₂O and analyzed by Agilent 2100 Bioanalyzer for library size distribution. The library was then quantitated by Picogreen Assay (Life Technologies) and sequenced with Illumina HiSeq2000 within either paired-end or single-end modes.

TABLE 1
Oligo Sequences

| | SEQ | |
|---|---|---|
| | ID | |
| Name | NO: | Sequence |
| L-20 | 1 | NNNNNNNNNNNNNNNNNNNN |
| L-40 | 2 | NNNNNNNNNNUGAGGUAGUAGGUUGUAUAGNNNN |
| | | NNNNNN |
| L-60 | 3 | NNNNNNNNNNUGAGGUAGUAGGUUGUAUAGUGAG |
| | | GUAGUAGGUUGUAUAGNNNNNNNNNN |
| RD-40-N9 | 4 | NNNNUGAGGUAGUAGGUUGUAUAGUGAGGUAGUA |
| | | GNNNNN |
| Adapter | 5 | GATCGGAAGAGCACACGTCT |
| oligo 1 | | |
| Adapter | 6 | ACACTCTTTCCCTACACGACGCTCTTCCGATCT |
| oligo 2 | | |
| PCR Primer | 7 | AATGATACGGCGACCACCGAGATCTACACTCTTT |
| 1 | | CCCTACACGACGCTCTTCCGATCT |
| PCR Primer | 8 | CAAGCAGAAGACGGCATACGAGATCGTGATGTGA |
| 2-1 | | CTGGAGTTCAGACGTGTGCTCTTCCGATCT |

TruSeq Library Preparation.

Libraries were prepared using Illumina TruSeq kit by following the instructions provided in the kit. The quantities and PCR cycles used in the library preparation are described in the Examples.

RC-Seq and RC2-Seq Library Data Analysis.

Each pair of the obtained raw reads first underwent merging to get the full-length sequence of the original cDNA molecules using the program FLASH. The minimum overlapping length was set at 10 nt. The merged paired-end reads were kept in one file, while the unmerged paired-end reads were kept in two different files. Next, each merged paired-end read as well as each first read in the unmerged reads file underwent repeating unit extraction using a Perl script (see Appendix A). In this script, maximum error number is set at 10% of the length of a repeating unit. After the repeating unit is identified for each read, a read expansion script is used to expand the repeating unit by moving one base at a time from its 5′ end to its 3′ end so the number of reads generated in the group is equal to the number of bases of the repeating unit (see Appendix B). Each read in the group was then aligned to hg19 using TopHat2 using the default parameters (maximum 2 errors). All the alignment data were combined into one file for each sample and sorted based on the read identity (see Appendix C and Appendix D). The read which was uniquely aligned and had the highest alignment score read in the group was chosen as the only one to represent the original RNA sequence in a SAM format (see Appendix E). The SAM file was converted to BAM file for visualization. For CLIP-Seq data, the BAM file is the input file for Mi-CLIP to further search the binding sites of a protein.

Expanding-then-Aligning Approach Validation by Simulation.

To examine whether the above described expanding-then-aligning approach is valid in reproducing the original RNA sequence, a computational simulation was carried out. In the simulation, five groups of reads with different length were generated: 20 nt, 40 nt, 60 nt, 80 nt, and 100 nt. For each group, 5000 reads were randomly selected computationally. The original genomic location of each read was recorded during generation. For each group, 5000 reads were aligned to hg19 using TopHat2 and only uniquely aligned reads and their alignments were retained. By comparison with their original genomic locations, the percentages of correctly uniquely aligned reads and incorrectly uniquely aligned reads were calculated. Then an expanding-then-aligning approach described above (see the computer program listings Appendix A-E) was used for each read in each group. Similarly, the percentages of correctly and incorrectly uniquely aligned reads were calculated.

Clustering of CLIP-Seq Tags.

The SAM format alignment files for each condition were pooled. For each condition, duplicate reads that have the same mapping coordinates (including strand) were collapsed to a single tag. Tags overlapping by at least one nucleotide were grouped together to form CLIP clusters, and those not overlapping with any other tags were discarded. The number of T->C mutations on each base was counted for all genomic regions covered by CLIP clusters.

Identifying Enriched Regions.

The Hidden Markov Model (HMM) (Rabiner, 1989) was used to determine the enriched regions from observed tag counts. First of all, CLIP clusters were divided into bins of 10 bp. If xₜ^{(k)} is the total tag count in the t-th bin of k-th cluster, then cluster k could be represented as a vector of tag counts:

\vec{x}^{(k)}=(x₁^{(k)},x₂^{(k)}, . . . , x_{T}_{ₖ}^{(k)}).

This HMM has two states:

$$\{ \begin{matrix}{I}_{t }^{( k )} = 0 & if bin t is non \text{-} enriched \\ {I}_{t}^{( k )} = 1 & if bin t is enriched\end{matrix} .$$

Since Poisson distribution is a popular model to fit count data (Xing et al., 2012; Zhang et al., 2008), the observed tag counts were modeled by a two-component Poisson mixture model:

$$\{ \begin{matrix}{X}_{t}^{( k )} \sim Poisson ( {λ}_{0} ) \vert {I}_{t}^{( k )} = 0 \\ {X}_{t}^{( k )} \sim Poisson ( {λ}_{1} ) \vert {I}_{t}^{( k )} = 1\end{matrix} ,$$

given state Iₜ^{(k)}. So the emission probability can be written as

$$Pr ( {X}_{t}^{( k )} = x \vert {λ}_{0} , {λ}_{1} , ω ) = ( 1 - ω ) \frac{{λ}_{0}^{x} {e}^{- {λ}_{0}}}{x !} + ω \frac{{λ}_{1}^{x} {e}^{- {λ}_{1}}}{x !} , ( {λ}_{0} < {λ}_{1} ) ,$$

where ω is the proportion of enriched bins in the CLIP clusters. The transition matrix Π is a 2×2 matrix, where element π_{r,s} is the transition probability

Pr(Iₜ^{(k)}=s|Iₜ₋₁^{(k)}=r).

The λ₀, λ₁ and ω parameters were estimated from the observed data using method of moments (Harter, 1975), the HMM algorithm was applied, and then the Viterbi algorithm (Viterbi, 1967) was used to infer the hidden states Iₜ^{(k)}, namely the enriched vs. non-enriched bins. Finally, each run of adjacent enriched bins were concatenated into one enriched region.

Identify Reliable Binding Sites.

A second round of HMM was used to identify reliable binding sites. This HMM has two states:

$$\{ \begin{matrix}{D}_{b}^{( n )} = 0 & if base pair b is not a binding site \\ {D}_{b}^{( n )} = 1 & if base pair b is a binding site\end{matrix} .$$

Each concatenated enriched region was divided into a series of bins of 1 bp for single-nucleotide resolution. Let (m_{b}⁽ⁿ⁾, x_{b}⁽ⁿ⁾) be the number of mutation and total tag counts in the b-th base pair of the n-th enriched region. The observed number of mutations M_{b}⁽ⁿ⁾ given the tag count X_{b}⁽ⁿ⁾ given D_{b}⁽ⁿ⁾ was modeled by

$$\{ \begin{matrix}{M}_{b}^{( n )} \vert {X}_{b}^{( n )} \sim ZIB ( {p}_{0} , {X}_{b}^{( n )} , ϕ ) \vert {D}_{b}^{( n )} = 0 \\ {M}_{b}^{( n )} \vert {X}_{b}^{( n )} \sim Bin ( {p}_{1} , {X}_{b}^{( n )} ) \vert {D}_{b}^{( n )} = 1\end{matrix} .$$

Here, a zero inflated binomial distribution (ZIB) (Hall, 2000) with probability p₀, size X_{b}⁽ⁿ⁾, and inflation parameter φ was used to model the background mutations, such as random sequencing errors at non-binding sites (D_{b}⁽ⁿ⁾=0), and a binomial distribution with probability p₁ and size X_{b}⁽ⁿ⁾ was used to model the cross-linking induced mutations at RNA-protein binding sites (D_{b}⁽ⁿ⁾=1). So, the emission probability is written as:

$$Pr ( {M}_{b}^{( n )} = m \vert {X}_{b}^{( n )} = x , {p}_{0} , {p}_{1} , θ , ϕ ) = ( 1 - θ ) [ ϕ I ( m = 0 ) + ( 1 - ϕ ) ( \begin{matrix}x \\ m\end{matrix} ) {{p}_{0}^{m} ( 1 - {p}_{0} )}^{x - m} ] + θ [ ( \begin{matrix}x \\ m\end{matrix} ) {{p}_{1}^{m} ( 1 - {p}_{1} )}^{x - m} ] ,$$

where θ is the proportion of binding sites in enriched regions. The parameters were estimated as follows: first, two modes, \hat{f}₁ and \hat{f}₂, were assumed in the density plot of mutation rates (m/x), of which \hat{f}₁ corresponds to the probability for success of the background ZIB component and \hat{f}₂ corresponds to the probability of success for the binomial component. A parameter c, specified according to experience, was chosen so that \hat{f}₁<c<\hat{f}₂. The bins with a mutation ratio m/x<c were used to estimate p₀ and φ for ZIB distribution using the method of moments, and the remaining bins were used to estimate p₁ for the binomial distribution. Again, the HMM algorithm was applied and the Viterbi algorithm (Viterbi, 1967) was used to infer the hidden states and the probability of being a reliable binding site Pr(D_{b}⁽ⁿ⁾=1|\vec{X},\vec{M}) for each base pair.

Implementation of the MiClip Algorithm.

This algorithm was implemented in an R package, MiClip. Part of the package was written in Perl to improve the efficiency and flexibility in handling large sequencing data. The package source, user manual, and vignette have been documented on CRAN (on the world wide web at http://cran.r-project.org). A user-friendly web-based interface was also developed for MiClip. This interface was built on the Galaxy platform Goecks et al., 2010; Blankenberg et al., 2010; Giardine et al., 2005), and all the analysis parameters were automatically saved to ensure the reproducibility of the data analysis.

### Example 2

### RC-Seq and RC2-Seq: Highly Sensitive Sequencing Methods for Small RNAs

The inventors developed a straightforward methodology that could be readily adopted by researchers accustomed to standard RNA-seq protocols and platforms, achieve greater than 100-fold improvement in sensitivity for small (<200 nucleotide) nucleotide (nt) fragments, and demonstrate at least a similar quality of sequencing output relative to standard methods. The developed method avoids the challenges inherent in intermolecular ligation while working at temperatures that reduce secondary structure and allow more uniform recognition of fragment termini.

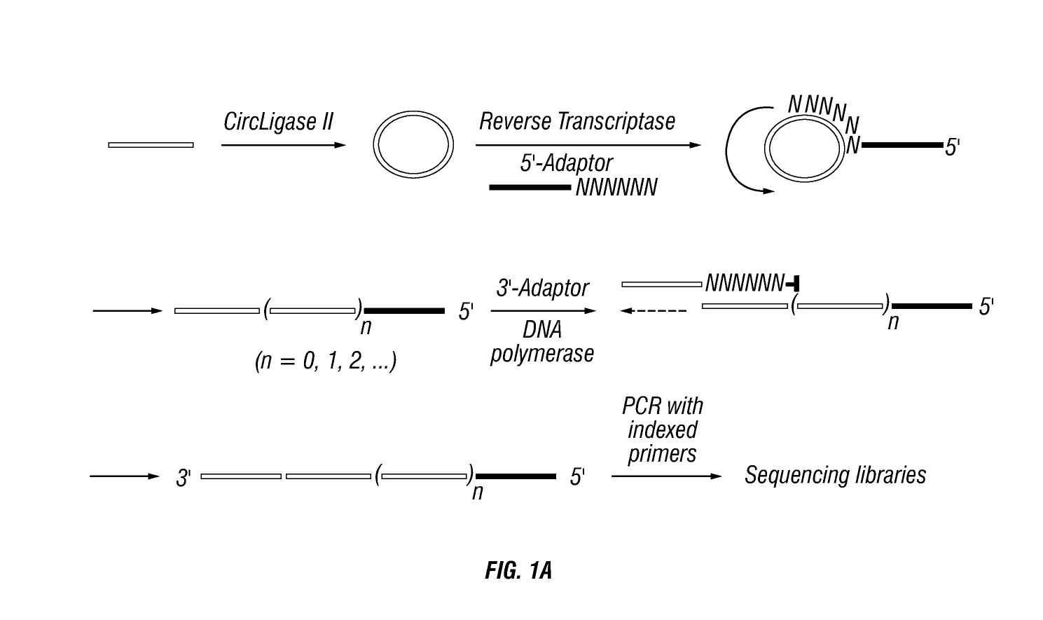

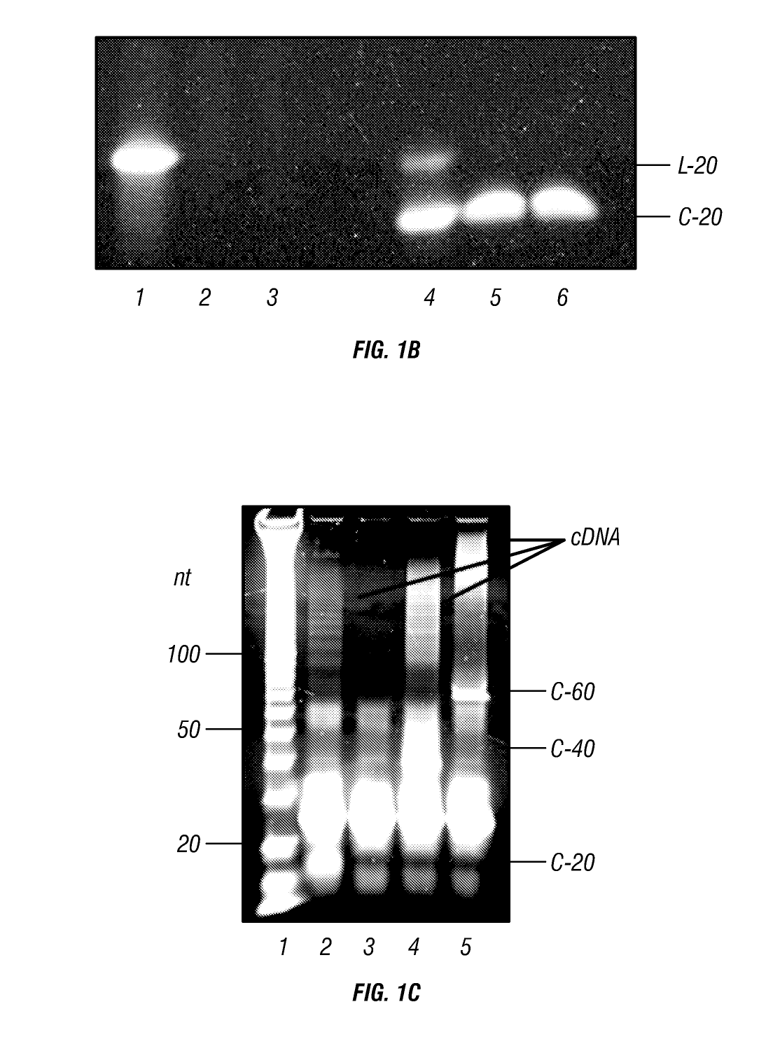

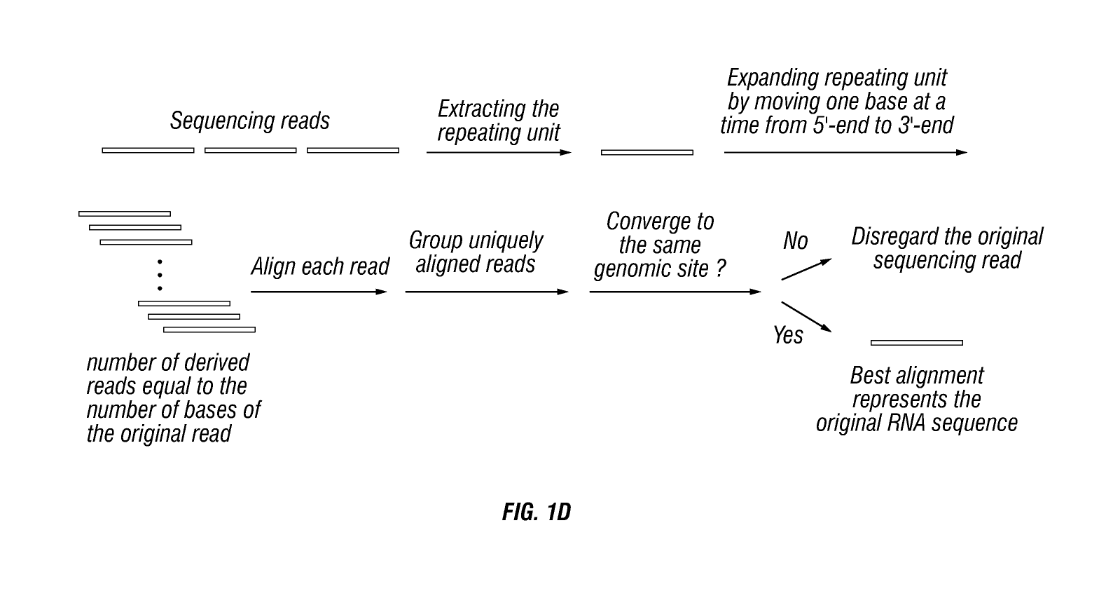

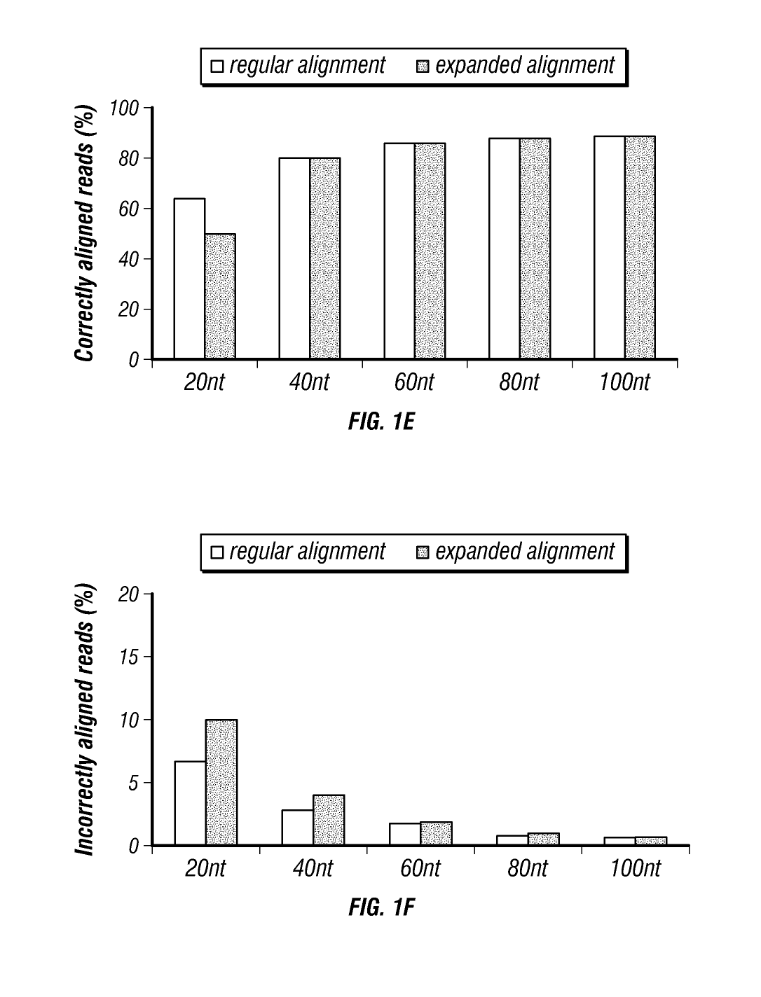

The inventors exploited the principle that intramolecular reactions are more favorable than analogous intermolecular reactions by developing a methodology that uses RNA self-circularization (FIG. 1A). The inventors used adaptor oligonucleotides for cDNA synthesis that associate by base-pairing rather than ligation. This recognition by simple base-pairing increases the efficiency of association needed for efficient template preparation because it does not require two successful ligations. This strategy alleviates the limitations inherent in methods that employ intramolecular ligations by requiring less RNA (picogram amounts) and yielding greater sequencing depth.

The potential for intramolecular ligation was investigated by optimizing conditions for RNA circularization efficiency using synthetic 20 nt, 40 nt, and 60 nt linear oligonucleotides. CircLigase II was chosen for the ligation step because it is a thermostable enzyme that efficiently catalyzes circularization of DNA templates possessing 5′-phosphate and 3′-hydroxyl groups (Polidoros et al., 2006). The circularization reaction was carried out at 60° C. for 1 h using CircLigase II (FIG. 1B, lanes 1 and 4). No adaptor oligonucleotides were required during this step. Because CircLigase II is thermostable, elevated temperatures were used to reduce the potential for intramolecular structure at the termini and increase the likelihood that the termini would be accessible for ligation. Following circularization, any remaining linear RNA can be removed by RNase R treatment at 37° C. for 15 min (FIG. 1B, lanes 5-6).

Ligation conditions were optimized and it was found that the conversion rate to circularized product was over 80% for the three differently-sized oligonucleotides. In the ligations, the circularized RNA was the only product detected, likely because the intramolecular reaction was heavily favored. By contrast, standard TruSeq methods that use adaptor RNAs yield multiple products (Viollet et al., 2011). Any residual linear RNA was removed by treatment with RNase R (Suzuki et al., 2006), an exonuclease that specifically degrades single-stranded linear RNA from the 3′ end.

The circularized RNA was used as a template for reverse transcription to create a library for RNA-seq. To prime the reverse transcription step and install a 5′ primer recognition sequence for subsequent PCR, tagged random primers were used that hybridize to the template by Watson-Crick base-pairing. Increasing the number of randomized bases from 6 to 10 did not increase the RT efficiency; thus, tagged random hexamers were used for subsequent experiments. Then, the mixture of circularized RNA and hybridized primer was treated with reverse transcriptase to convert the RNA into complementary DNA (cDNA) (FIG. 1C). Multiple reverse transcriptases were tested and it was found the Superscript II was the most efficient at using circular RNA as a template. Because the template is circular and subject to rolling circular amplification (Polidoros et al., 2006), multiple copies of the fragment sequence within the cDNA were an expected outcome and were dealt with by developing modified protocols for computational analysis (see, Example 2).

After obtaining linear cDNA, a tagged oligonucleotide was hybridized to the linear cDNA and DNA polymerase was used to extend the DNA strand and create a product with two primer recognition sites that could be used for PCR. The tagged primer was blocked at the 3′ position so that it was only capable of introducing a site at the 3′ terminus of the cDNA. Then, PCR was performed with one primer binding the 3′ tag and a second primer binding the 5′ tag.

Following PCR amplification, the crude sequencing library was purified by PAGE to obtain products of the appropriate size (200-400 base-pairs) or by Ampure XP magnetic beads designed to separate duplex DNA from single-stranded primers. After purification, the quality of library was confirmed by Bioanalyzer and quantitated by Pico-Green assay.

The purified sample was analyzed by RNA sequencing using an Illumina HiSeq 2000 sequencer. Paired-end sequencing was used because pair-ended sequencing allows better coverage of molecules greater than 100 base-pairs. Sequencing was performed in duplicate and all conditions for sequencing were standard. Sequencing libraries were bar-coded to permit running multiple samples per lane.

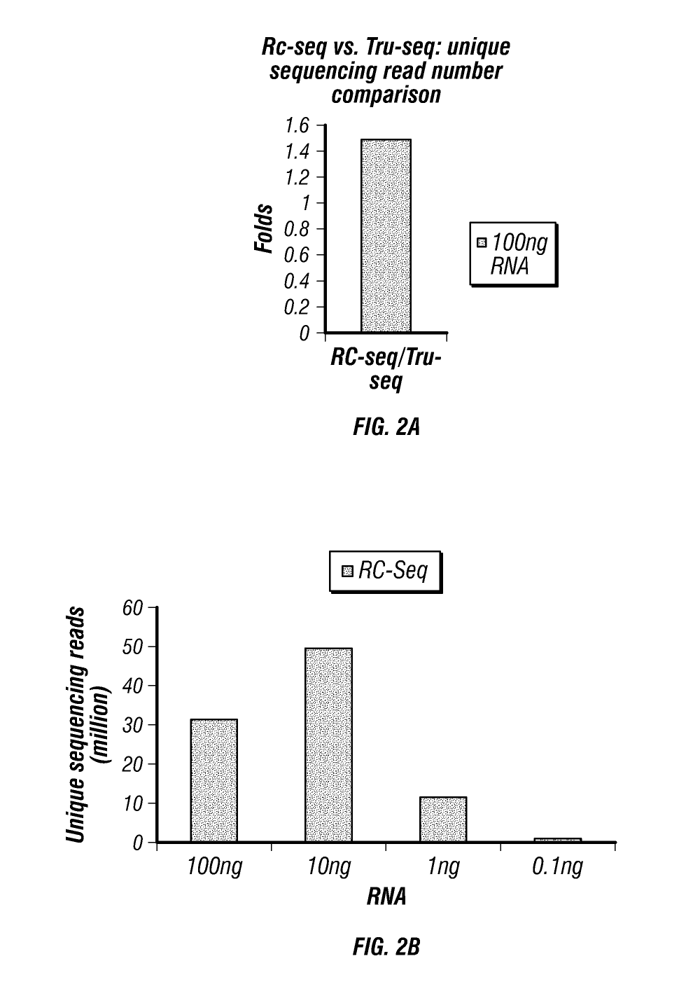

To gauge the sensitivity of this RNA circularization-based RNA-seq library preparation approach, libraries were prepared using both commercially available Illumina TruSeq small RNA kits and the present method. Both methods were performed using random linear 40 nt synthetic RNA with 10¹² maximum unique sequences as starting material (L-40). One library was generated using the TruSeq library with 100 ng of RNA as the starting material. Four libraries were generated using the present method, with 100 ng, 10 ng, 1 ng, and 0.1 ng of 40 nt RNA as the starting material. The present method generated more reads for the 100 ng library than did the TruSeq method (FIG. 2A). The read quality generated by both methods was also compared and the present method was found to have identified more unique sequences than the TruSeq method by 50%. To test the quantity threshold at which RC-Seq will fail to produce sufficient unique sequencing reads, a series of experiment trials were carried out by reducing the starting RNA quantity from 100 ng, to 10 ng, 1 ng, and 0.1 ng. Sequencing results indicated that using as low as 1 ng of starting RNA, RC-Seq can still generate more than 10 million unique reads (FIG. 2B). However, when the starting RNA was at 0.1 ng, the unique sequences identified by RC-Seq decreased dramatically to less than 1 million.

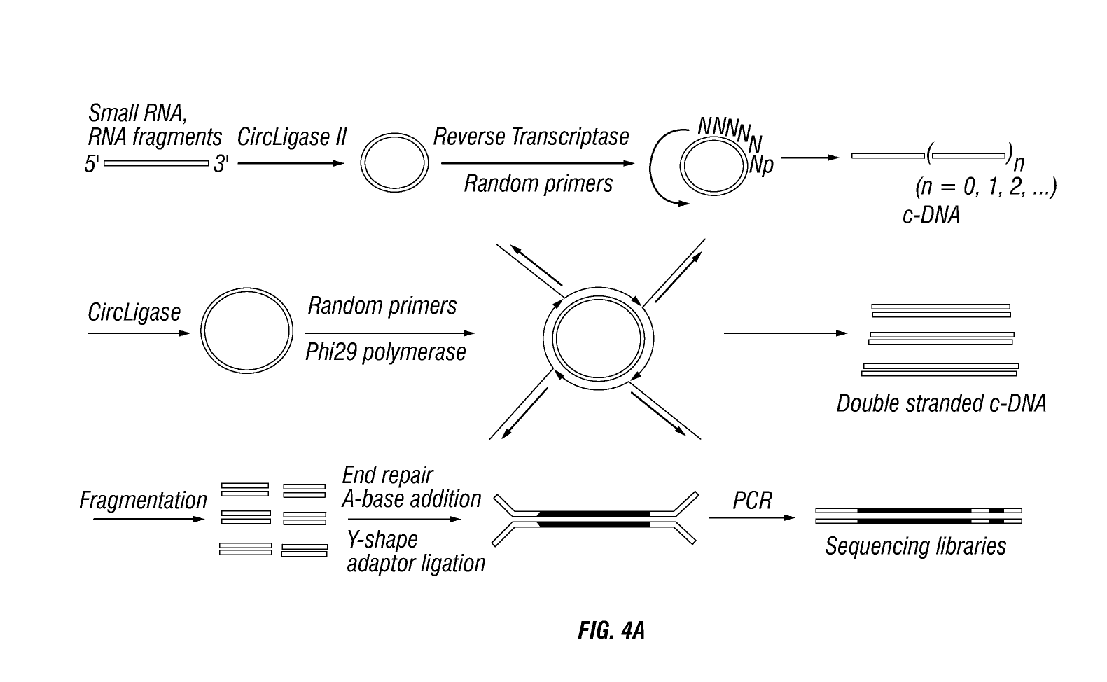

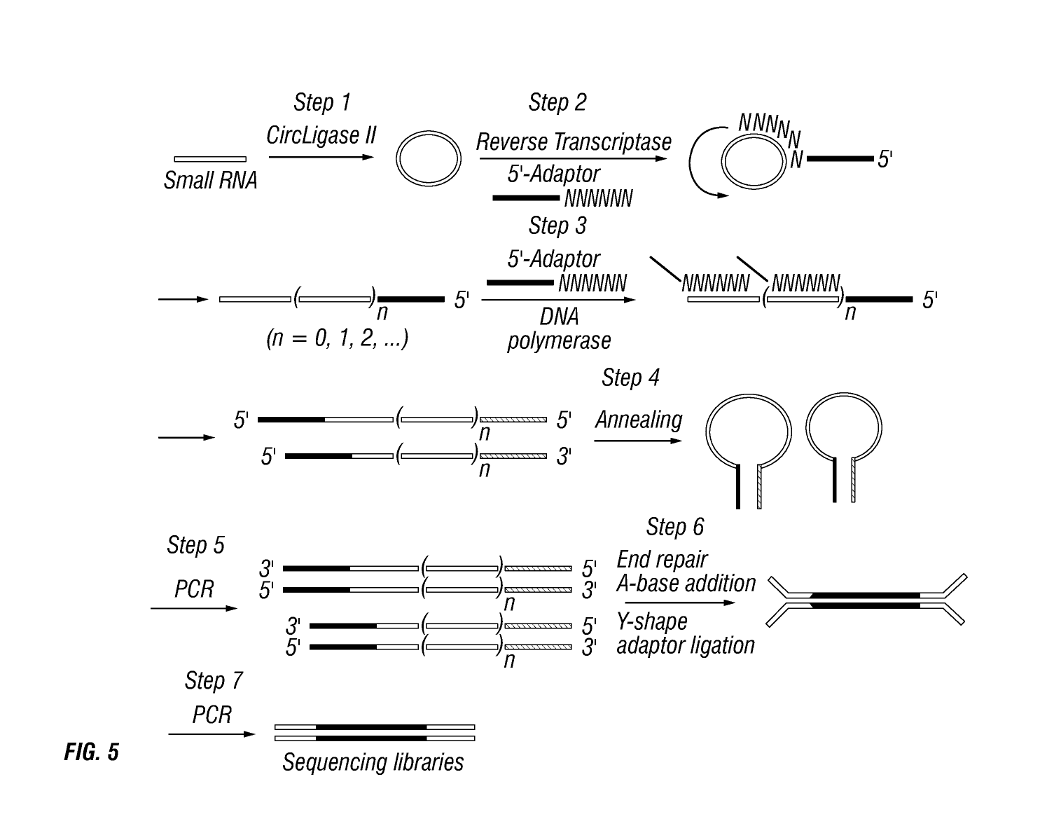

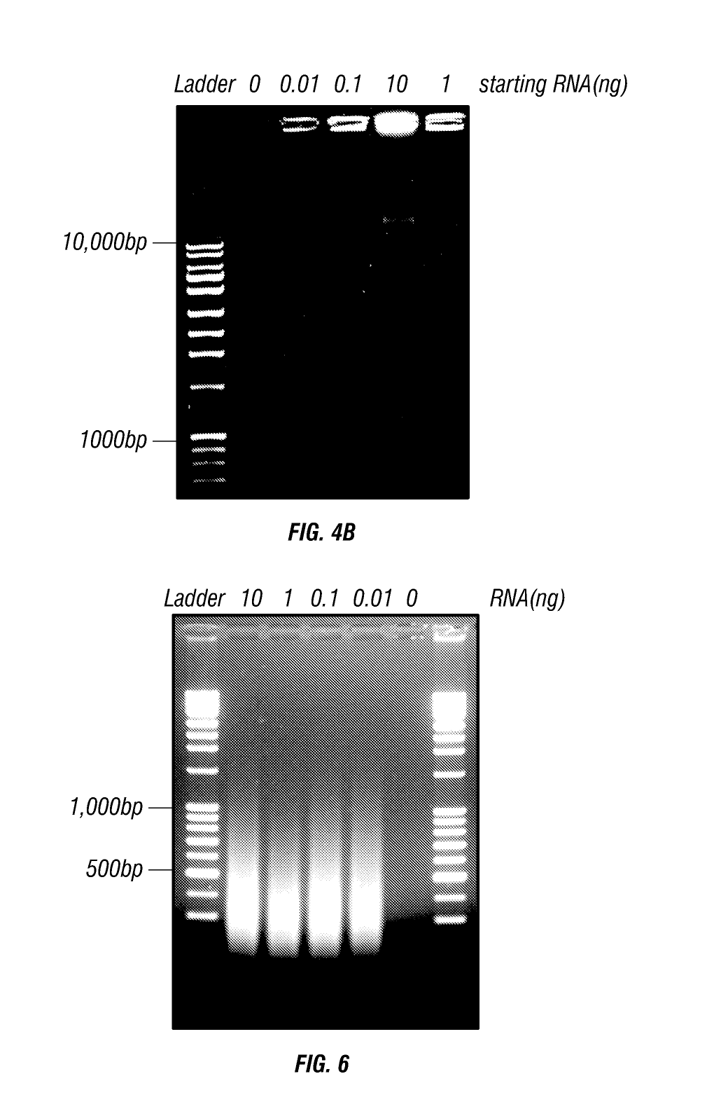

In order to improve the sensitivity of the method, the library preparation method was expanded to include two circularization steps (RC2-Seq): one for the original RNA sample and a second for the reverse transcribed single-stranded cDNA (FIG. 4A). Following the second circularization step, random primers were used to prime DNA polymerase reactions to generate double-stranded cDNA, which was then fragmented by sonication. Following fragmentation, a standard DNA-seq protocol comprising end-repair A base ligation and Y-shaped adaptor ligation followed by PCR amplification will be used to prepare sequencing libraries. As low as 10 pg of RNA (a single-cell amount) was successfully amplified for sequencing library preparation (FIG. 4B). Sequencing data will show comparable sequencing sensitivity and depth from RC2-Seq when using 100 ng to 100 pg of starting RNA.

### Example 3

### RC-Seq and RC2-Seq: Expanding-then-Aligning Algorithm

Before the outcome of present RNA-seq method could be interpreted, it was necessary to develop new computational tools. The tools generated can be run on any UNIX operating system (FIG. 1D; computer program listings Appendix A-E). The ligation method used in the RC-Seq and RC2-Seq protocols introduces multiple tandem repeats and existing software was not able to efficiently locate the original sequences. As all the reads contained repeating units, and the first step was to identify the repeating unit as a single sequence. Depending on where the tagged random hexamer primers hybridized, the repeating unit could differ even if derived from the same parent sequence. To recover the original RNA fragment or miRNA sequence, the 3′ and 5′ ends were computationally shifted in one base increments to create a family of sequences. Each member of the family was tested for its ability to align with a reference genome, and the one with the highest alignment score was taken to represent the original RNA sequence.

To validate the algorithm as a tool for analyzing data, a simulation was carried out to compare the output from the present approach to results generated by standard alignment methods. In the simulation, groups of 5000 sequences were randomly selected from the human genome (hg19) having lengths of 20 nt, 40 nt, 60 nt, 80 nt, and 100 nt. The original genomic location was recorded for each “read,” or sequence. To evaluate how well standard methods process this data, TopHat216 was used to align these reads to hg19. Uniquely aligned reads were retained for subsequent analysis. For the 20 nt group, only ˜60% of all randomly selected reads were uniquely aligned (FIG. 1E). The percentage increased to 80% or higher when the read length increased to 40 nt or longer. The incorrectly aligned rates were also calculated for each group, with 20 nt having a 6% error rate, 40 nt having 3%, and 60 nt or longer having less than 2% (FIG. 1F).

Next, the expanding-then-aligning approach was applied to the same randomly generated sequences. This approach performed similarly well as the standard method (FIG. 1E). The incorrect alignment rates were extremely close when the read length was 40 nt or longer (FIG. 1F). These simulation data demonstrate that the expanding-then-aligning approach correctly recovers the original RNA sequence in the human genome.

### Example 4

### Sequencing of Human AGO2-Associated RNA Using RC-Seq

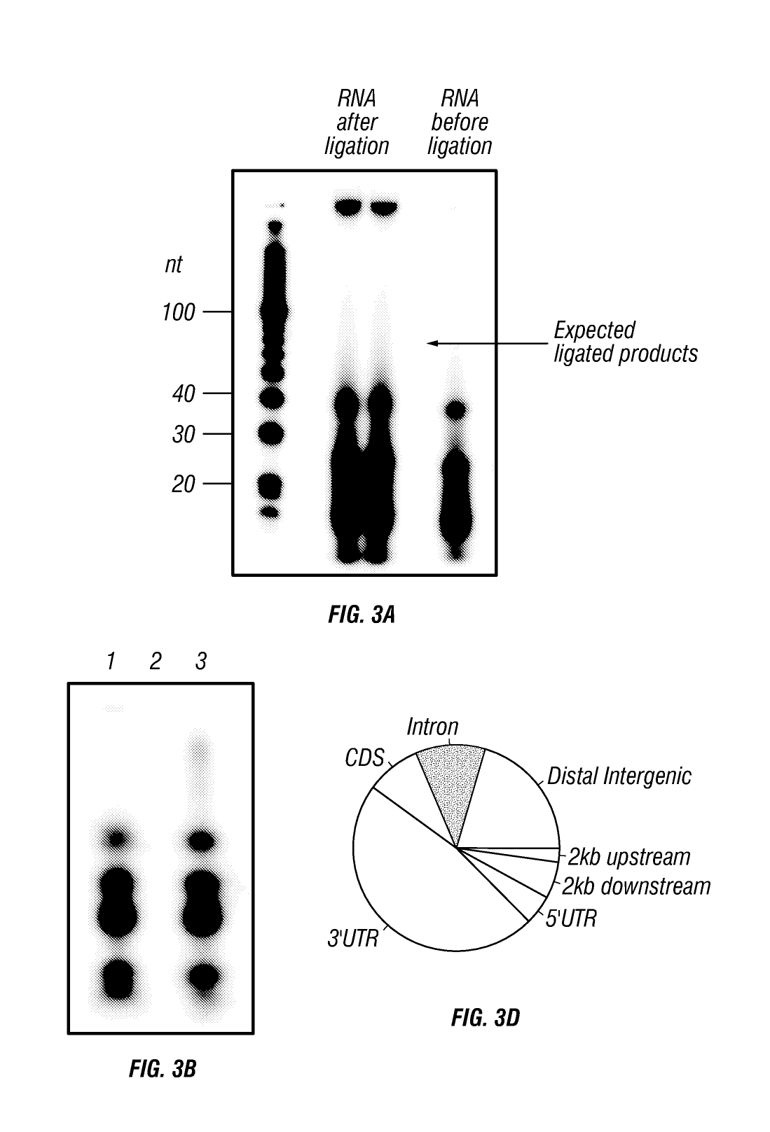

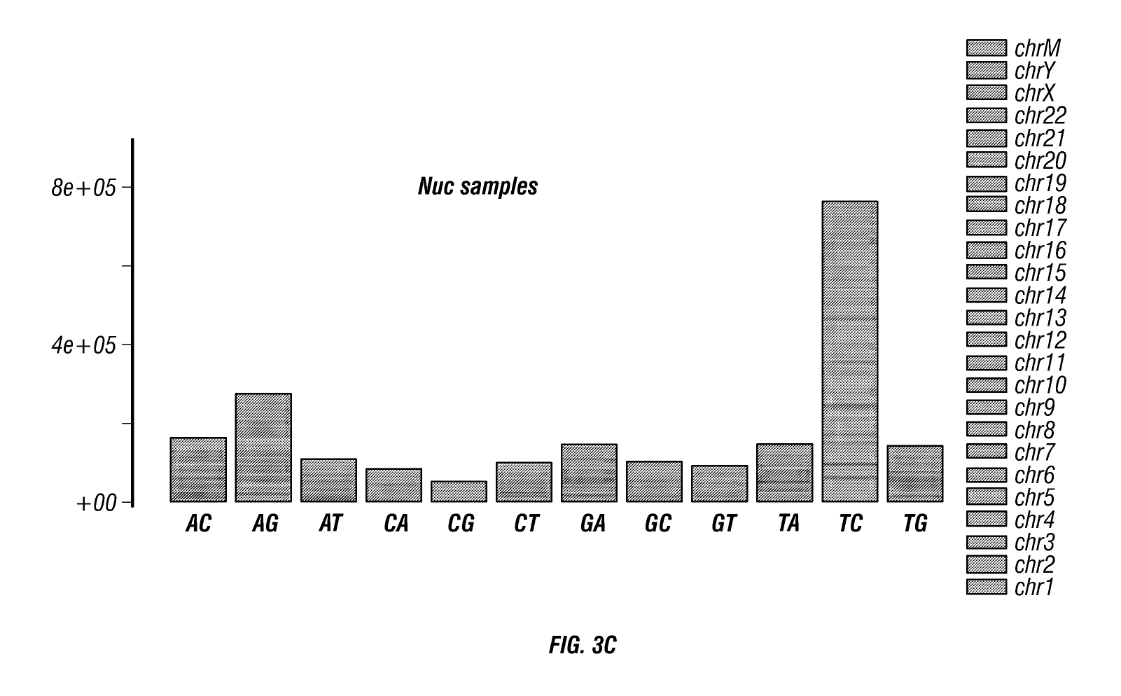

To further demonstrate deeper sequence coverage ability and higher sensitivity of this method, the RC-Seq method was used to sequence human AGO2-associated RNA obtained following photoactivatable-ribonucleoside-enhanced crosslinking and immunoprecipitation (PAR-CLIP) (Hafner et al., 2010). PAR-CLIP is a highly specific and stringent protocol for identifying RNA species associated with an RNA-binding protein. In this protocol, RNase I was used to partially digest the RNA bound to AGO2. Thus, only RNA bound within the AGO2 binding pocket was protected and thus could be detected. The clipped RNA obtained was determined to be on the picogram scale and RNA sizes ranged from 50 nt to 20 nt. The traditional adaptor-RNA ligation and polyA-tailing approaches did not work efficiently as an expected size shift was not observed following the ligation (FIG. 3A). The attempt to make traditional sequencing libraries thus failed.

The present method was applied to check how much RNA can be circularized. It was reasoned that after circularization, 5′-P³² labeled RNA would no longer be accessible to RNase R degradation (Epicentre, 37° C., 10 min, 20 U RNase R). It was found that ˜75% of the RNA was converted into circular RNA (FIG. 3B). This was a dramatic increase in terms of sequencing depth over the traditional method. Following cDNA production and PCR amplification, the library was sequenced and the data analyzed. First, the raw data were subjected to the expanding-then-aligning approach to generate uniquely aligned data. The sequencing data showed a dominant T-to-C mutation over others, a characteristic feature of PAR-CLIP-generated sequencing data (FIG. 3C) (Hafner et al., 2010; Konig et al., 2012). Then, using Mi-CLIP (Wang et al., 2014), software specializing in searching protein binding sites in RNA from CLIP-seq datasets, over 1000 significant clusters were identified as enriched regions and binding sites. Subsequent genomic annotation showed that more than 50% of the clusters localized in gene 3′-untranslated regions (3′-UTR) (FIG. 3D). These results are consistent with what has been reported about human AGO2 function, as AGO2/miRNA complexes are known to interact with mRNA in this region to regulate translation (Chi et al., 2009; Hafner et al., 2010; Kumar et al., 2011).

### Example 5

### Improved Method Version of RC-Seq

FIG. 5 shows a scheme for an improved version or RC-Seq. Steps 1 and 2 were the same as those in RC-Seq, in which RNA was circularized and cDNA was produced with appropriate reverse transcriptase (as described before). The cDNA was purified by DNA Clean & Concentrator-5 kit (Zymo Research) and eluted with 10 μl of nuclease-free water. The purified cDNA was then linearly amplified with a DNA polymerase, either BST DNA polymerase, large fragment or BST 2.0 DNA polymerase (New England Biolabs). The linear amplification was composed of 5 cycles. In the first cycle, 1 μl of 25 μM of 5′-adaptor, 1.8 μl of 10× reaction buffer, 1 μl of 10 mM dNTP mix, along cDNA were heated 94° C. for 3 min, then cooled to 58° C. for 20 s, then stored on ice immediately. From the second to the sixth cycles, 1 μl of DNA polymerase was added in each cycle and the mixture was heated slowly from 10° C. to 65° C., stayed at 65° C. for 2 min, then 94° C., then 58° C., then on ice. The final reaction mixture was purified with DNA Clean & Concentrator-5 kit (Zymo Research) and eluted with 23 μl of water. The eluted duplex DNA was further processed with end-base repair, A-addition and adaptor ligation, and a final PCR to produce an indexed sequencing library, ready for paired-end Illumina sequencing.

As shown in FIG. 6, RC3-Seq successfully generating high quality libraries with as low as 10 picograms (pg) of input small RNA. The input RNA was 40 nt randomized synthetic RNA, RD-40-N9 (Table 1). The inventors have determined that a library size from 200 to 500 bp is ideal for standard paired-end sequencing. Generally speaking, a single cell contains at least 10 pg of total RNA, which contains long RNA and small-sized RNA, such as miRNAs.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Adiconis et al., Comparative analysis of RNA sequencing methods for degraded or low-input samples, Nat. Methods, 7:623-629, 2013.
Blankenberg et al., Galaxy: a web-based genome analysis tool for experimentalists, Curr. Protoc. Mol. Biol., Chapter 19:Unit 19.10.1-21, 2010.
Borges-Rivera et al., Deep sequencing of the small RNA transcriptome of normal and malignant human B cells identifies hundreds of novel microRNAs, Blood, 116:e118-127, 2010.
Chakravarty et al., RNA ligase RtcB splices 3′-phosphate and 5′-OH ends via covalent RtcB-(histidinyl)-GMP and polynucleotide-(3′)pp(5′)G intermediates, PNAS, 109:6072-6077, 2012.
Chi et al., Argonaute HITS-CLIP decodes microRNA-mRNA interaction maps, Nature, 460:479-486, 2009.
Chu et al., Involvement of argonaute proteins in gene silencing and activation by RNAs complementary to a noncoding transcript at the progesterone receptor promoter, Nucleic Acids Res., 38:7736-7748, 2010.
Esther et al., Deep sequencing of RNA from immune cell-derived vesicles uncovers the selective incorporation of small non-coding RNA biotypes with potential regulatory functions, Nucl. Acids Res., 18:9272-9285, 2012.
Giardine et al., Galaxy: a platform for interactive large-scale genome analysis, Genome Res., 15:1451-1455, 2005.
Goecks et al., Galaxy: a comprehensive approach for supporting accessible, reproducible, and transparent computational research in the life sciences, Genome Biol., 11:R86, 2010.
Hafner et al., Transcriptome-wide identification of RNA-binding protein and microRNA target sites by PAR-CLIP, Cell, 141:129-141, 2010.
Hall, Zero-inflated Poisson and binomial regression with random effects: A case study, Biometrics, 56:1030-1039, 2000.
Harter, Probabilistic Approach to Automatic Keyword Indexing. 1. Distribution of Specialty Words in a Technical Literature, Journal of the American Society for Information Science, 26:197-206, 1975.
Jensen and Darnell, CLIP: crosslinking and immunoprecipitation of in vivo RNA target of RNA-binding proteins, Methods Mol. Biol., 488:85-98, 2008.
Kim et al., TopHat2: accurate alignment of transcriptomes in the presence of insertions, deletions and gene fusions, Genome Biology, 14:R36, 2013.
Konig et al., Nature Rev. Genet., 2012.
Kumar et al., miR-ID: A novel, circularization-based platform for detection of microRNAs, RNA, 17:365-380, 2011.
Licatalosi et al., HITS-CLIP yields genome-wide insights into brain alternative RNA processing, Nature, 456(7221):464-469, 2008.
McCormick et al., Experimental design, preprocessing, normalization and differential expression analysis of small RNA sequencing experiments, Silence, 2:2, 2010.
Mercer et al., The Human Mitochondrial Transcriptome, Cell, 146:645-658, 2011.
Ozsolak and Milos, RNA sequencing: advances, challenges and opportunities. Nature Rev. Genet., 12:87-98, 2011.
Polidoros et al., Rolling circle amplification-RACE: a method for simultaneous isolation of 5′ and 3′ cDNA ends from amplified cDNA templates, BioTechniques, 41:35, 2006.
Rabiner, A Tutorial on Hidden Markov-Models and Selected Applications in Speech Recognition, Proceedings of the IEEE, 77:257-286, 1989.
Ramsköld et al., Full-length mRNA-Seq from single-cell levels of RNA and individual circulating tumor cells, Nat. Biotechnol., 30:777-782, 2012.
Shalek et al., Single-cell transcriptomics reveals bimodality in expression and splicing in immune cells, Nature, 498:236-240, 2013.
Skarnes et al., A public gene trap resource for mouse functional genomics, Nat. Genet., 36:543-544, 2004.
Suzuki et al., Characterization of RNase R-digested cellular RNA source that consists of lariat and circular RNAs from pre-mRNA splicing, Nucl. Acids Res., 34:63, 2006.
Tang et al., mRNA-Seq whole-transcriptome analysis of a single cell, Nat. Meth., 6:377-382, 2009.
Tang et al., Development and applications of single-cell transcriptome analysis, Nat. Methods, 8:S6-S11, 2011.
Ule et al., CLIP identifies Nova-regulated RNA networks in the brain, Science, 302(5648):1212-1215, 2003.
Ule et al., CLIP: a method for identifying protein-RNA interaction sites in living cells, Methods, 37(4):376-386, 2005.
Viollet et al., T4 RNA Ligase 2 truncated active site mutants: improved tools for RNA analysis, BMC Biotechnology, 11:72, 2011.
Viterbi, Error Bounds for Convolutional Codes and an Asymptotically Optimum Decoding Algorithm, IEEE Transactions on Information Theory, 13:260-269, 1967.
Wang et al., Model-based approach to identify binding sites in CLIP-Seq data, Submitted, 2014.
Xing et al., A novel Bayesian change-point algorithm for genome-wide analysis of diverse ChIPseq data types, J. Vis. Exp., e4273, 2012.
Xue et al., Genetic programs in human and mouse early embryos revealed by single-cell RNA sequencing, Nature, 500:593-597, 2013.
Yeo et al., An RNA code for the FOX2 splicing regulator by mapping RNA-protein interactions in stem cells, Nat. Struct. Mol. Biol., 16(2):130-137, 2009.
Zhang et al., Model-based analysis of ChIP-Seq (MACS), Genome Biol., 9:R137, 2008.
Zhang and Darnell, Mapping in vivo protein-RNA interactions at single-nucleotide resolution from HITS-CLIP data, Nat. Biotechnol., 29:607-614, 2011.
Zhuang et al., Structural bias in T4 RNA ligase-mediated 3′-adapter ligation, Nucl. Acids Res., 40:e54, 2012.

1. A method of preparing an RNA sample for sequencing comprising:
(a) obtaining a sample comprising RNA molecules;
(b) self-ligating each RNA molecule in the sample to form circular RNA;
(c) hybridizing a first set of random primers to the circular RNA;
(d) extending the first set of random primers hybridized to the circular RNA to form cDNA;
(e) self-ligating the cDNA to form a circular cDNA;
(f) hybridizing a second set of random primers to the circular cDNA; and
(g) extending the second set of random primers hybridized to the circular cDNA to form double-stranded cDNA.

2-28. (canceled)

29. A method of preparing an RNA sample for sequencing comprising:
(a) obtaining a sample comprising RNA molecules;
(b) self-ligating each RNA molecule in the sample to form circular RNA;
(c) hybridizing a first set of random primers to the circular RNA, wherein the first set of random primers comprises a 5′ adaptor of known sequence;
(d) extending the first set of random primers hybridized to the circular RNA to form cDNA;
(e) hybridizing a second set of random primers to the cDNA, wherein the second set of random primers comprises a 3′ adaptor of known sequence; and
(f) extending the second set of random primers hybridized to the cDNA.

30. The method of claim 29, further comprising (g) amplifying the cDNA of step (f) thereby producing a sequencing library.

31. The method of claim 30, wherein the amplifying comprises performing PCR with indexed primers.

32. The method of claim 29, wherein self-ligating each RNA comprises treating the RNA sample with CircLigase II, RtcB, or T4 RNA ligase.

33. The method of claim 29, wherein step (d) comprises performing reverse transcription.

34. The method of claim 30, further comprising (h) obtaining sequencing data for the sequencing library.

35. The method of claim 34, further comprising (i) identifying the original RNA sequence by aligning to a reference.

36. The method of claim 29, wherein the random portions of the first set of random primers comprising a 5′ adaptor of known sequence and second set of random primers comprising a 3′ adaptor of known sequence are random hexamers.

37. The method of claim 29, wherein the adaptor portions of the first set of random primers comprising a 5′ adaptor of known sequence and second set of random primers comprising a 3′ adaptor of known sequence are different.

38. The method of claim 29, wherein the first set of random primers comprising a 5′ adaptor of known sequence and second set of random primers comprising a 3′ adaptor of known sequence are nuclease resistant RNA primers.

39. The method of claim 29, wherein the RNA molecules in step (a) are single-stranded.

40. The method of claim 29, wherein the RNA sample comprises less than 1 ng of RNA.

41. The method of claim 29, wherein the RNA sample comprises or consists essentially of 10-500 pg of RNA.

42. The method of claim 29, wherein the RNA sample comprises or consists essentially of less than 250 pg of RNA.

43. The method of claim 29, wherein the RNA sample comprises or consists essentially of less than 100 pg of RNA.

44. The method of claim 29, wherein the RNA sample comprises or consists essentially of about 10 pg of RNA.

45. The method of claim 29, wherein the RNA sample comprises RNA obtained from a single cell.

46. The method of claim 29, wherein the RNA sample comprises or consists essentially of RNA molecules of 20 to 750 nt in length.

47. The method of claim 29, wherein the RNA sample comprises or consists essentially of RNA molecules less than 200 to 500 nt in length.

48. The method of claim 29, wherein the RNA sample comprises or consists essentially of RNA molecules of 100-200 nt in length.

49. A kit comprising a single-stranded RNA ligase, a reverse transcriptase, and a DNA polymerase.

50-59. (canceled)


SEQUENCE LISTING (8 sequences)

SEQ ID NO: 1 - DNA, 20 bp, Artificial sequence
  Synthetic oligo
nnnnnnnnnnnnnnnnnnnn

SEQ ID NO: 2 - DNA, 40 bp, Artificial sequence
  Synthetic oligo
nnnnnnnnnnugagguaguagguuguauagnnnnnnnnnn

SEQ ID NO: 3 - DNA, 60 bp, Artificial sequence
  Synthetic oligo
nnnnnnnnnnugagguaguagguuguauagugagguaguagguuguauagnnnnnnnnnn

SEQ ID NO: 4 - DNA, 40 bp, Artificial sequence
  Synthetic oligo
nnnnugagguaguagguuguauagugagguaguagnnnnn

SEQ ID NO: 5 - DNA, 20 bp, Artificial sequence
  Synthetic oligo
gatcggaagagcacacgtct

SEQ ID NO: 6 - DNA, 33 bp, Artificial sequence
  Synthetic oligo
acactctttccctacacgacgctcttccgatct

SEQ ID NO: 7 - DNA, 58 bp, Artificial sequence
  Synthetic oligo
aatgatacggcgaccaccgagatctacactctttccctacacgacgctcttccgatct

SEQ ID NO: 8 - DNA, 64 bp, Artificial sequence
  Synthetic oligo
caagcagaagacggcatacgagatcgtgatgtgactggagttcagacgtgtgctcttccgatct